(19) United States
(12) Patent Application Publication
(10) Pub. No.: US 2025/0249069 A1
(43) Pub. Date: Aug. 7, 2025

(54) High-Density Nutritional Composition with Plant Polyphenols

(71) Applicant: Goode Health LLC, Greenwich, CT (US)

(72) Inventors: Matthew J. Wiant, Fairfield, CT (US); Michael Edward Glick, Galena, OH (US)

(21) Appl. No.: 18/433,942

(22) Filed: Feb. 6, 2024

(51) Int. Cl. A61K 38/16; A61K 9/00; A61K 31/07; A61K 31/355; A61K 31/375; A61K 31/4375; A61K 31/592; A61K 31/7048; A61K 31/718; A61K 31/736; A61K 33/06; A61K 33/26; A61K 33/30; A61K 36/28; A61K 36/45; A61K 36/54; A61K 36/55; A61K 36/752; A61K 36/82; A61K 36/8962; A61K 36/9066; A61K 36/9068; A61P 3/06; A61P 29/00

(52) U.S. Cl. CPC A61K 38/168; A61K 9/0095; A61K 31/07; A61K 31/355; A61K 31/375; A61K 31/4375; A61K 31/592; A61K 31/7048; A61K 31/718; A61K 31/736; A61K 33/06; A61K 33/26; A61K 33/30; A61K 36/28; A61K 36/45; A61K 36/54; A61K 36/55; A61K 36/752; A61K 36/82; A61K 36/8962; A61K 36/9066; A61K 36/9068; A61P 3/06; A61P 29/00

(57) ABSTRACT

A high-density nutritional composition is described comprising nutrients (protein, polyphenol, fiber, vitamins and mineral) and fillers, in a ratio of nutrients to fillers of at least 1.5:1. A nutritional combination is also described, where the nutritional composition can also be combined with one or more antioxidant and micronutrient boosters, to provide additional nutrients and health benefits, such as improving an individual's vascular health (e.g., blood circulation, blood vessel flexibility, cholesterol levels, and blood pressure), inflammation, immunity, cognitive function or reducing aging.

### BACKGROUND

The Western diet contains an excess of empty calories, calories that do not contribute to the nutrient density of the diet. Existing nutritional compositions contain significant amounts of fillers or empty calories and low amounts of nutrients. There is a need to minimize empty calories and consume more nutrient-dense foods.

### SUMMARY

A high-density nutritional composition is described comprising nutrients (protein, polyphenol, fiber, vitamins and mineral) and fillers, in a ratio of nutrients to fillers of at least 1.5:1. The nutritional composition contains a low amount of empty calories and fillers (e.g. corn syrup, sugar, other caloric sweeteners, simple carbohydrates, gums, undesirable fats) and a high amount of desirable nutrients, in particular plant-derived nutrients such as proteins, fiber, vitamins, minerals, anti-oxidants and polyphenols. The nutritional composition provides key vitamins, minerals, and antioxidants missing in the diets of most (95% +) people. The present disclosure also relates to a nutritional combination, where the nutritional composition can also be combined with one or more antioxidant and micronutrient boosters, to provide additional nutrients and health benefits, such as improving an individual's vascular health (e.g., blood circulation, blood vessel flexibility, cholesterol levels, and blood pressure), inflammation, immunity, cognitive function or reducing aging.

### DETAILED DESCRIPTION

A description of example embodiments follows.

### Base Compositions

The present disclosure relates generally to compositions comprising a high-density of nutrients with a ratio of nutrients to fillers of at least about 1.5:1, or at least about 60% of calories from nutrients and at most about 30% of calories from fillers; wherein the composition can be consumed in a single or divided daily serving.

The 1.5:1 ratio of nutrients to fillers (e.g. at least about 60% nutrients) is far higher than any commercial product previously available on the market. Currently available nutritional products range from 0% to 50% caloric composition of nutrients, with the top selling products like ENSURE™ and BOOST™ containing less than 20% of caloric contribution from nutrients. Orgain Plant protein and Ka'Chava even contain 37% and 53% empty calories, respectively.

The high-density nutritional composition helps supplement nutritional deficiencies in an individual's diet as well as providing a source of powerful antioxidants and functional benefits associated with polyphenols. The high-density nutrients present in the composition uniquely reduce, eliminate, or replace the presence of fillers and accordingly reduce or eliminate empty calories. The nutritional compositions act to curb the appetite and reduce or eliminate cravings by providing the body with nutrients which it would otherwise crave due to their short supply. The compositions described herein can comprise plant-derived ingredients, particularly plant-based proteins, plant-based fiber, plant-based polyphenols, and other naturally sourced ingredients. The compositions of the disclosure can further comprise or consist of one or more ingredients such as, but not limited to, protein, fiber, vitamins, minerals, antioxidants, flavorants, co-factors, such as enzymes (e.g., proteases), desirable fats, complex carbohydrates, sugar substitutes, and lesser amounts of sugars and simple carbohydrates which may be naturally present in certain food ingredients.

A “high-density” nutritional composition, as used herein, is a composition in which the proportion of nutrients on a dry weight percentage basis exceeds the dry weight percentage of fillers that contribute empty calories by a ratio of 1.5:1. The ingredients selected for inclusion in the nutritional composition individually possess nutritional value for desired functional benefits, but when combined in the compositions of the disclosure, they provide a single serving source of nutrients that can harness the collective functional benefits of the combination, without unnecessarily increasing caloric intake, empty calories and unnatural ingredients. High-density can be achieved in a single serving by selecting ingredients that serve multiple purposes, for example, carob and chia seeds are excellent sources of protein, fiber and polyphenols. An advantage of the high-density compositions of the disclosure is that they can also be customized for a particular functional benefit or personalized according to the end-user's nutritional needs and desired functional benefits, such as by adding additional nutrient-dense ingredients, referred to herein as an “antioxidant booster,” to a “base” composition. In an embodiment, the “base” composition is a high-density nutritional composition of the disclosure, without an antioxidant booster(s) present. When the antioxidant booster(s) is/are incorporated into the base composition, the resulting composition is referred to herein as a “nutritional combination”.

Nutrients, as defined herein, are compounds found in foods that are essential for growth and metabolic functioning, and include, but are not limited to, protein, plant fiber, vitamins, minerals, co-factors, such as enzymes (e.g., proteases), polyphenols (e.g., flavonoids, phenolic acids), and antioxidants. In embodiments, the nutrients are derived from natural sources, particularly plant sources. Nutrients selected for incorporation into the compositions of the disclosure should not contribute to the empty calorie content of the composition.

As used in this disclosure, “fillers” which usually contain empty calories are defined to mean the ingredients (and their associated calories) that do not contribute a positive effect to the nutrient content of the nutritional composition of the disclosure. They can provide a source of energy (e.g. calories) or some other non-nutritive feature. Sources of empty calories include, but are not limited to, added saccharides (e.g., added sugar, corn syrup, maltodextrin, sucrose, dextrose, lactose, sugars, and other ingredients that are mostly saccharides), polyols (e.g., erythritol sorbitol, maltitol, etc), gums, grain flours, saturated fats, and also other lower caloric chemicals typically added to foods for other purposes besides nutrition (e.g., synthetic sweeteners, flavor enhancers, colorings or whiteners, preservatives, bulking agents, flow agents, anti-foaming agents, anti-caking agents). Because these sources of empty calories do not contribute to the nutritional value of the compositions of the disclosure, they should be present in low amounts or avoided altogether. In embodiments, it may be desirable to include low amounts of empty calories sources to achieve desired features, such as organoleptic properties, taste, stability, consumer acceptability, among other reasons. If present, the amounts should be within the ratios and teachings of the disclosure to ensure a high-density composition.

A high-density nutritional composition may provide a serving size of from about 10 g to about 300 g, about 10 g to about 200 g, about 10 g to about 100 g, about 20 g to about 90 g, or about 25 g to about 60 g, on a dry weight basis. Other serving sizes on a dry weight basis are contemplated and such amounts can be ascertained by the formulator based on the consumer's needs and functional benefits of the composition. A serving size can also be ascertained by the consumer.

In some embodiments, the percentage of nutrients in a high-density nutritional composition is from about 55 wt. % to about 100 wt. %, from about 60 wt. % to about 100 wt. %, from about 60 wt. % to about 90 wt. %, from about 65 wt. % to about 85 wt. %, from about 70 wt. % to about 80 wt. %, from about 70 wt. % to about 75 wt. %, or from about 75 wt. % to about 80 wt. %.

On a serving basis, the compositions of the disclosure focus on delivery of important nutrients that are supplemented to the diet. In some embodiments, the ratio of nutrients to filler (on a dry weight basis) in the nutritional composition is at least about 1.5:1, at least about 2:1, at least about 2.5:1, at least about 3:1, at least about 4:1, at least about 5:1, at least about 6:1, at least about 7:1, at least about 8:1, at least about 9:1, or at least about 10:1. Other ratios of nutrient to filler (on a dry weight basis) are contemplated and such ratios can be ascertained by the formulator based on the consumer's needs and functional benefits of the composition.

In other embodiments, the amount of filler in the nutritional composition is from about 2 wt. % to about 50 wt. %, from about 10 wt. % to about 45 wt. %, from about 0 wt. % to about 40 wt. %, from about 15 wt. % to about 40 wt. %, from about 20 wt. % to about 35 wt. %, from about 20 wt. % to about 30 wt. %, from about 25 wt. % to about 30 wt. %, or from about 20 wt. % to about 25 wt. %.

An example embodiment of a high-density nutritional composition comprises or consists of: a ratio of nutrients to filler that exceeds about 1.5:1; at least 1 source of plant polyphenols that collectively provide a level of polyphenols of from about 50 mg to 200 mg (e.g., polyphenol levels are present in a single or divided daily serving of the composition from about 50 mg to about 200 mg); vitamins and minerals present in an amount of from about 0 g to about 6 g; plant protein present in an amount of from about 5 g to 35 g (e.g., single plant source or combination of plant sources); carbohydrates present in an amount of from about 1 g to about 15 g plant-based fiber present in an amount of from about 2.5 g to about 24 g; and fat present in an amount of from about 0 g to about 6 g. The example embodiment can be provided as a multi-serving composition to supply a week, a month or other allocation of servings. Alternatively, the composition can be a single serving or packaged as a single serving ready-to-drink beverage.

In aspects of this example embodiment, the level of polyphenols is from about 50 mg to 200 mg per serving size of about 25 g to about 60 g on a dry weight basis. In some aspects, the level of polyphenols is about 50 mg to about 60 mg per serving size of about 28.8 g. In another aspect, the composition provides calories in an amount of from about 2.5 calories per gram of composition to about 3.5 calories per gram of composition.

In yet another aspect, the example composition provides a level of vitamins and minerals which are missing in the diets of most Americans: vitamin B9, zinc, vitamin A, magnesium, vitamin C, vitamin E, potassium, vitamin D, iron, or calcium, or a combination thereof. Preferably, each of these vitamins are collectively provided in the example embodiment as a “vitamin booster”. Levels of vitamins added to the composition can be readily ascertained by the formulator. For example, the composition provides a level of vitamin D of from about 0.6 mg per gram of composition to about 0.8 mg per gram of composition.

In other aspects, the example composition provides less than about 0.5 g of sugar, less than about 0.5 g of saturated fat, and/or less than about 0.5 g of trans fat. In embodiments, it may be desirable to provide a composition that is low in sugar, sugar-free or sweetened by sugar substitutes, particularly naturally derived sugar substitutes.

A specific, non-limiting example described herein is a vanilla flavored high-density composition comprising vitamin booster (vitamin B9, zinc, vitamin A, magnesium, vitamin C, vitamin E, potassium, vitamin D, iron, and calcium), plant protein(s) (e.g., pea, chia, rice, fava or any combination of these), soluble fiber, optional sweetening agent(s), such as monk fruit. To this “base” composition, antioxidant booster(s) can be optionally added, such as bilberry, green tea, isoquercetin, echinacea, black garlic, bergamot, cinnamon, ginger, turmeric, berberine and zinc, individually or in combinations, depending upon desired functional benefits. In embodiments, a serving size of about 25g to about 60 g on a dry weight basis will provide about 50 mg to about 200 mg of polyphenols and fillers in an amount of about 5% to about 25% of total calories (or by total weight).

Another specific, non-limiting example described herein is a chocolate flavored high-density composition comprising vitamin booster (vitamin B9, zinc, vitamin A, magnesium, vitamin C, vitamin E, potassium, vitamin D, iron, and calcium), plant protein(s) (e.g., pea, chia, rice, fava or any combination of these), soluble fiber, optional sweetening agent(s), such as monk fruit, cocoa powder and carob (provides source of flavorant and polyphenols). To this “base” composition, antioxidant boosters can be optionally added, such as bilberry, green tea, isoquercetin, echinacea, black garlic, bergamot, cinnamon, ginger, turmeric, berberine and zinc, individually or in combinations, depending upon desired functional benefits. In embodiments, a serving size of about 25 g to about 60 g on a dry weight basis will provide about 50 mg to about 200 mg of polyphenols and fillers in an amount of about 5% to about 25% of the total calories (or total weight).

An unsweetened version of these specific, non-limiting examples is also contemplated.

The presence of ingredients which have multiple benefits allows for the use of natural antioxidants which in turn allows the composition to be storage stable without the need for preservatives.

### Polyphenols

As defined herein, “polyphenols” are chemical compounds or molecules that are characterized by having at least one aromatic ring with one or more hydroxyl groups. Polyphenols can be synthetically produced or found naturally occurring in plant, vegetable, fruit and other sources, for example. Plant polyphenols, as used herein, are naturally occurring polyphenols and are derived from plant source material. Extracted polyphenols from these sources provide concentrated polyphenols in higher levels than can be otherwise consumed from nature. For example, natural blueberries may contain polyphenol levels of about 5 mg/gram of blueberries, but bilberry extracts contain up to about 500 mg/gram. In order to optimize the polyphenol nutritional value of the compositions of the disclosure, extracts will be used. In some embodiments, when the polyphenol level is naturally robust in a plant source, the plant source itself may be included in the composition. Cocoa is an example of a source of high-level polyphenols that does not need to be extracted, with polyphenol levels as high as about 80 mg/grams of cocoa.

Polyphenols used in the nutritional compositions can include, but are not limited to, a flavonoid, phenolic acid, stilbene, or lignan, or a combination thereof. Examples of flavonoids include, but are not limited to, anthocyanins, catechins, chalcones, dihydrochalcones, dihydroflavonols, flavanols, flavanones, flavones, flavonols, or isoflavonoids, or a combination thereof. A preferred flavonoid is isoquercetin.

Other example polyphenols can include, but is not limited to, alkylmethoxyphenols, alkylphenols, curcuminoids, furanocoumarins, hydroxybenzaldehydes, hydroxybenzoketones, hydroxycinnamaldehydes, hydroxycoumarins, hydroxyphenylpropenes, methoxyphenols, naphtoquinones, phenolic terpenes, tannin, tannic acid, and tyrosols.

As discussed in more detail, a polyphenol can be extracted from plants, plant parts, plant cells, or plant products. Examples of plant parts and sources of plant polyphenols include, but are not limited to, bark, flower, petal, stem, stalk, tuber, root, fruit, berry, seed, nut, leaf. Examples of plant products include, but are not limited to, pomace juice, pulp, skin, mash, paste, and slurry. Examples of plants include, but are not limited to, blueberry, cranberry, grape, peach, plum, pomegranate, soy, red wine, black tea, green tea. In some embodiments, the polyphenols are extracted from multiple plants, plant parts, or plant products. In some embodiments, the polyphenol extract is combined from multiple plants, plant parts, or plant products. In other embodiments, sources of plant polyphenols comprise bilberry, green tea, isoquercetin, echinacea, carob, ginger, turmeric, garlic or monk fruit, cinnamon and bergamot or a combination thereof.

Polyphenols may be extracted from any suitable source, such as, e.g., foods rich in polyphenols, including, but not limited to, cloves, peppermint, star anise, cocoa powder, oregano, celery seed, black chokeberry, dark chocolate, flaxseed meal, black elderberry, chestnut, common sage, rosemary, spearmint, common thyme, lowbush blueberry, blackcurrant, capers, black olive, highbush blueberry, hazelnut, pecan nut, soy flour, plum, green olive, sweet basil, curry, powder, sweet cherry, globe artichoke heads, blackberry, roasted soybean, milk chocolate, strawberry, red chicory, red raspberry, coffee, filter, ginger, whole grain hard wheat flour, prune, almond, black grape, red onion, green chicory, common thyme, refined maize flour, soy, tempeh, whole grain rye flour, apple, spinach, shallot, lemon verbena, black tea, red wine, green tea, soy yogurt, yellow onion, soy meat, whole grain wheat flour, pure apple juice, pure pomegranate juice, extra-virgin olive oil, black bean, peach, pure blood orange juice, cumin, pure grapefruit juice, white bean, Chinese cinnamon, pure blond orange juice, broccoli, redcurrant, soy tofu, pure lemon juice, whole grain oat flour, apricot, caraway, refined rye flour, asparagus, walnut, potato, Ceylan cinnamon, parsley, nectarine, curly endive, marjoram, red lettuce, chocolate beverage with milk, quince, endive (escarole), soy milk, pure pummelo juice, rapeseed oil, pear, soybean sprout, green grape, carrot, vinegar, soy cheese, white wine, and rosé wine.

Polyphenols may not only be derived from suitable plant sources (e.g. plant extracts) but they can be synthesized. Polyphenols may be synthesized via a suitable combination of chemical, biochemical, or biotechnological methods known in the art. In some embodiments, the polyphenols are extracted from natural sources and subsequently modified via chemical, biochemical, or biotechnological methods. In some embodiments, the synthesized or modified polyphenol chemical structures are not found in nature. Examples of chemical modifications include but are not limited to methylation, hydroxylation, prenylation, glycosylation, dimzerization, polymerization. Examples of glycosylation include but are not limited to glucosides, galactosides, arabinosides, rhamnosides.

In some embodiments, the polyphenol is selected from hydroxybenzoic acids, hydroxycinnamic acids, hydroxyphenylacetic acids, hydroxyphenylpropanoic acids, and hydroxyphenylpentanoic acids. In some embodiments, the polyphenol is isoquercetin or berberine.

In other embodiments, the nutritional composition comprises alkaloids. For non-limiting example, the alkaloid is a plant alkaloid. In an aspect, the alkaloid is berberine.

In other embodiments, the nutritional composition comprises plant-based or spice-based ingredients, or both plant-based and spice-based ingredients, including and not limited to ginger, garlic, cinnamon, bergamot, flax seeds, turmeric, bilberry, green tea extract, and echinacea. In an aspect, the turmeric is a sustained release turmeric, such as turmeric with MERIVA™ bioavailable coating (Indena, Milan, Italy). In another aspect, the garlic is black garlic. In another aspect, the plant-based ingredient may also be a particular compound derived from a plant, such as berberine. In another aspect, the spice-based ingredient may also be a compound derived from a spice, such as curcumin.

In some embodiments, the nutritional composition comprises at least one source, at least two sources, at least three sources, at least four sources, at least five sources, at least 12 sources or more, between 1-5 sources, between 1-4 sources, between 1-3 sources, between 1-2 sources, between 2-5 sources, between 3-5 sources, between 2-4 sources, between 3-4 sources, between 4-5 sources, or between 2-3 sources of plant polyphenols that collectively provide a level of polyphenols of from about 0.1 wt. % to about 5 wt. %, from about 0.1 wt. % to about 3 wt. %, from about 0.5 wt. % to about 4.5 wt. %; from about 0.5 wt. % to about 4 wt. %; from about 0.5 wt. % to about 3.5 wt. %; from about 0.5 wt. % to about 3 wt. %; from about 1 wt. % to about 3 wt. %, from about 1 wt. % to about 2.5 wt. %, from about 1.5 wt. % to about 2.5 wt. %, from about 1.5 wt. % to about 2 wt. %, from about 1 wt. % to about 1.5 wt. %, from about 0.1 wt. % to about 0.7 wt. %, or from about 0.1 wt. % to about 0.3 wt. %. In some non-limiting examples, using a broader spectrum of plant sources provides a wider profile of polyphenols with a wider range of antioxidant and anti-inflammation attributes.

In some embodiments, the nutritional composition collectively provides a level of polyphenols in a single daily dose or divided daily dose. In a non-limiting example, a nutritional composition of a serving size of from about 25 g to about 60 g on a dry weight basis provides a level of polyphenols from about 0 mg to about 200 mg, 50 mg to about 1000 mg, from about 200 mg to about 700 mg, or from about 300 mg to about 650 mg, in a single or divided dose.

### Protein, Fiber, Other Nutritionally Rich Ingredients

The nutritional composition can comprise protein, such as animal protein, plant protein, or a combination thereof. In embodiments, the protein is aqueous soluble protein. In embodiments, the composition is a plant-based composition and therefore comprises plant protein, and preferably aqueous soluble plant protein. In embodiments, the protein is present in an amount of from about 0 wt. % to about 95 wt. %, from about 0 wt. % to about 90 wt. %, from about 10 wt. % to about 90 wt. %, from about 10 wt. % to about 80 wt. %, from about 10 wt. % to about 70 wt. %, from about 20 wt. % to about 70 wt. %, from about 20 wt. % to about 60 wt. %, from about 30 wt. % to about 70 wt. %, from about 35 wt. % to about 65 wt. %, from about 40 wt. % to about 60 wt. %, from about 45 wt. % to about 55 wt. %, from about 45 wt. % to about 50 wt. %, from about 50 wt. % to about 55 wt. %, or from 52 wt. % to about 53 wt. %. In embodiments, the composition comprises proteins present in an amount of 52 wt. %. In other embodiments, a serving size from about 25 g to about 60 g on a dry weight basis of the nutritional composition provides protein in an amount of from about 5 g to about 35 g, from about 12 g to about 30 g, or from about 14 g to about 20 g.

In a preferred embodiment, the protein source is plant-based, such as from peas, chia seeds, rice, brown rice, fava beans, all or any combination of these, and provided in the compositions in amounts as discussed herein.

The nutritional composition can comprise fiber, such as a plant-based fiber (e.g. chia, fava bean, FIBERSOL®). For example, the fiber is present in an amount of from 0 wt. % to about 50 wt. %, from about 0 wt. % to about 40 wt. %, from about 10 wt. % to about 40 wt. %, from about 0 wt. % to about 30 wt. %, from about 5 wt. % to about 25 wt. %, from about 10 wt. % to about 20 wt. %, from about 15 wt. % to about 20 wt. %, or from about 17 wt. % to about 18 wt. %. In some embodiments, the composition comprises fiber present in an amount of about 17.4 wt. %. In an embodiment, the fiber is a prebiotic fiber or combination of prebiotic fiber. In other embodiments, a nutritional composition of a serving size of from about 25 g to about 60 g on a dry weight basis can provide fiber at a level of from about 2.5 g to about 24 g, from about 2 g to about 200 g, or from about 5 g to about 8 g.

The nutritional composition can contain one or more vitamins such as, but limited to. vitamin A, vitamin B, vitamin C, vitamin D, vitamin E, vitamin K, thiamine, riboflavin, pyridoxine, carotenoids, niacin, folic acid, pantothenic acid, biotin, choline, inositol, salts thereof, derivatives thereof, and combinations thereof. The combination of vitamins and use levels in the compositions of the disclosure can be readily ascertained by the formulator and will depend on the functional benefits of the composition, daily dosage levels, among other purposes. In some embodiments, the vitamin A is retinol, retinyl esters, or carotenoids, or a combination thereof. In some embodiments, the vitamin B is vitamin B1, vitamin B2, vitamin B3, vitamin B5, vitamin B6, vitamin B7, vitamin B9, or vitamin B12, or a combination thereof. In some embodiments, the vitamin D is vitamin D1, vitamin D2, vitamin D3, vitamin D4, or vitamin D5, or a combination thereof. In some embodiments, the vitamin K is vitamin K1 or vitamin K2, or a combination thereof.

In some embodiments, the nutritional composition provides a level of vitamin D of from about 0.4 mg per gram of composition to about 0.8 mg per gram of composition, from about 0.5 mg per gram of composition to about 0.8 mg per gram of composition, from about 0.6 mg per gram of composition to about 0.8 mg per gram of composition, from about 0.65 mg per gram of composition to about 0.75 mg per gram of composition, or from about 0.69 mg per gram of composition to about 0.73 mg per gram of composition. In some embodiments, the nutritional composition provides a level of vitamin D of about 0.69 mg per gram of composition. In other embodiments, the nutritional composition provides a level of vitamin D of about 0.73 mg per gram of composition.

A preferred combination of vitamins is provided as a “vitamin booster” and comprises or consists of vitamin B9, zinc, vitamin A, magnesium, vitamin C, vitamin E, potassium, vitamin D, iron, and calcium.

Example nutrients can be minerals such as, but not limited to, phosphorus, magnesium, iron, zinc, manganese, copper, sodium, iodine, calcium, potassium, molybdenum, chromium, selenium, chloride, boron, vanadium, and combinations thereof. The combination of minerals and use levels in the compositions of the disclosure can be readily ascertained by the formulator and will depend on the functional benefits of the composition, daily dosage levels, among other purposes.

The nutritional composition can comprise carbohydrates (e.g. fiber) from a single source or type, or combination of sources and types, within the ratios discussed in this disclosure. In embodiments, the carbohydrates are present in an amount of from 0 wt. % to about 50 wt. %, from about 10 wt. % to about 50 wt. %, from about 10 wt. % to about 40 wt. %, from about 3 wt. % to about 25 wt. %, from about 10 wt. % to about 30 wt. %, from about 20 wt. % to about 30 wt. %, or from about 24 wt. % to about 28 wt. %. In some embodiments, the composition comprises carbohydrates present in an amount of 24.3 wt. %. In other embodiments, the composition comprises carbohydrates present in an amount of 27.8 wt. %. In some embodiments, a nutritional composition of a serving size of from about 25 g to about 60 g on a dry weight basis provides carbohydrates in an amount of from about 1 g to about 100 g, about 1 g to about 50 g, about 1 g to about 15 g, about 1 g to about 10 g, or about 1 g to about 5 g.

Because the nutritional compositions of the disclosure are high-density and have low fillers, the calories of composition can be reduced compared to other commercially available compositions. For non-limiting example, the composition provides calories in an amount of from about 1 calorie per gram of composition to about 10 calories per gram of composition, from about 1 calorie per gram of composition to about 5 calories per gram of composition, from about 2 calories per gram of composition to about 4 calories per gram of composition, or from about 2.5 calories per gram of composition to about 3.5 calories per gram of composition. In other embodiments, the nutritional composition provides calories in an amount of about 2.8 calories per gram of composition. In other embodiments, the nutritional composition provides calories in an amount of about 3.1 calories per gram of composition.

In some embodiments, the nutritional composition contains empty calories. For non-limiting example, between about 2% to about 50%, between about 10% to about 45%, between about 15% to about 54%, between about 20% to about 45%, between about 25% to about 40%, between about 30% to about 35% of the total amount of calories, between about 10% and 25%, between about 11% and about 13%, or between about 22% and about 23% of the total calories in the nutritional composition are attributed to empty calories.

In some embodiments, the nutritional composition provides fat, for non-limiting example, one or more of unsaturated fat, saturated fat, and trans fat. The fat may be, for non-limiting example, animal-based or plant-based. In some embodiments, the fat is plant-based. In other embodiments, the nutritional composition provides about 0 g of saturated fat. In other embodiments, the nutritional composition provides about 0 g of trans fat.

For non-limiting examples, the total fat content of the nutritional composition is in an amount of from about 0 g per gram of composition to about 1 g per gram of composition, about 0 g per gram of composition to about 0.5 g per gram of composition, about 0 g per gram of composition to about 0.4 g per gram of composition, about 0 g per gram of composition to about 0.3 g per gram of composition, about 0 g per gram of composition to about 0.2 g per gram of composition, about 0 g per gram of composition to about 0.1 g per gram of composition, or about 0.05 g per gram of composition to about 0.06 g per gram of composition. In some embodiments, the nutritional composition of a serving size of from about 25 g to about 60 g on a dry weight basis provides fat in an amount of from about 0 g to about 50 g, about 0 g to about 40 g, about 0 g to about 30 g, about 0 g to about 20 g, about 0 g to about 10 g, about 0 g to about 6 g, about 0 g to about 5 g, or about 0 g to about 3 g. In other embodiments, the nutritional composition provides fat present in an amount of from about 0 wt. % to about 30 wt. %, about 0 wt. % to about 20 wt. %, about 0 wt. % to about 10 wt. %, or about 0 wt. % to about 5 wt. %.

For non-limiting example, the total unsaturated fat content of the nutritional composition is in an amount of from about 0 g per gram of composition to about 1 g per gram of composition, about 0 g per gram of composition to about 0.5 g per gram of composition, about 0 g per gram of composition to about 0.4 g per gram of composition, about 0 g per gram of composition to about 0.3 g per gram of composition, about 0 g per gram of composition to about 0.2 g per gram of composition, about 0 g per gram of composition to about 0.1 g per gram of composition, or about 0.05 g per gram of composition to about 0.06 g per gram of composition.

In some embodiments, the nutritional composition provides sugars, for non-limiting example, one or more of added sugars and total sugars. In preferred embodiments, the nutritional composition provides about 0 g of total sugars. In an aspect, the nutritional composition provides about 0 g of added sugars. Sugar substitutes can be added to the nutritional composition.

For non-limiting example compositions, a composition of about 28.8 g per serving size can provide about 15 g of protein per serving, about 5 g of fiber per serving, about 2 g of vitamins and minerals per serving, and about 6.8 g of filler per serving, the composition providing 22 g of nutrients per serving, where the nutrients consists of protein, fiber, vitamins, and minerals, and wherein the vitamins and minerals comprise antioxidants and polyphenols. In other embodiments, the nutritional composition provides a level of any component of the composition, for non-limiting example, plant protein, fiber, and carbohydrates, in a single or divided dose.

### Antioxidant Boosters and Nutritional Composition Combinations

The present disclosure further relates to nutritional combinations (also referred to herein as nutritional composition combinations, high-density nutritional combinations, high-density nutritional composition combinations) comprising nutritional compositions of the disclosure and one or more antioxidant boosters incorporated into the nutritional composition. An antioxidant booster comprises one or a combination of natural antioxidant ingredients that provides functional benefits by virtue of their antioxidant and anti-inflammatory properties. Examples of functional benefits attributed to the antioxidant properties includes, but are not limited to, improving vascular health, reducing inflammation, enhancing immune system, improving cognitive function, reducing aging, and improving metabolism. In embodiments, the antioxidant booster comprises plant-based or spice-based ingredients, or both plant-based and spice-based ingredients, including and not limited to ginger, garlic, cinnamon, bergamot, flax seeds, turmeric, bilberry, green tea extract, and echinacea. In other embodiments, the antioxidant booster comprises chemical compounds derived from plants such as, but not limited to, berberine, isoquercetin, and curcumin.

An effective antioxidant booster to improve metabolism can comprise berberine, ginger, and cinnamon.

An effective antioxidant booster to enhance the immune system can comprise bilberry, green tea extract, isoquercetin, echinacea, vitamin C, and zinc.

An effective antioxidant booster to improve vascular health can comprise black garlic, bergamot, and flax seeds.

An effective antioxidant booster to reduce inflammation can comprise turmeric and isoquercetin. In an aspect, the turmeric is turmeric with MERIVA™ bioavailable coating.

Other boosters include, but are not limited to, an effective antioxidant booster to improve cognitive health, energy, gut health, skin health, and to provide anti-aging properties, prenatal support, calm and stress relief, and animal proteins. Non-limiting examples of ingredients and nutrients for the boosters include one or more of: taurine, caffeine, vitamin E, lutein, vitamin D, folate, magnesium, lavender, nicotinamide mononucleotide, resveratrol, collagen, and colostrum.

In some embodiments, an antioxidant booster provides an amount of polyphenols from about 2 wt % to about 6 wt %. The serving size of an antioxidant booster may be between about 0.25 g and about 3 g. An antioxidant booster may provide a level of polyphenols of about 20 mg to about 120 mg per serving.

### Uses of the Compositions, Composition Combinations and Functional Benefits

The disclosure is also directed to uses and methods for achieving one or more functional benefits in an individual in need thereof by consuming the nutritional composition or the nutritional combination as part of a nutritional regime or supplementation to the individual's diet, over a period of time. In a two-week clinical trial as discussed in the Examples section of this disclosure, preliminary data indicate that the nutritional composition results in a positive effect on blood lipid markers in subjects with respect to a control group. Cholesterol measures also displayed significant improvement, with total cholesterol decreasing by 14.5%, LDL cholesterol reducing by 18.7%, and Triglycerides dropping by 25.4%. A crucial marker of immune health and inflammation, C-reactive Protein, decreased by about 6% after two weeks of consumption of the nutritional composition. The presence of polyphenols and their use levels in the nutritional compositions and nutritional combination harnesses the functional benefits, particularly due to their antioxidant and anti-inflammatory properties. Many disease states may be prevented or improved in an individual by routine consumption of the nutritional compositions and nutritional combination of the disclosure.

Example functional benefits include, but are not limited to, sustained steady energy, improving vascular health, reducing inflammation, enhancing immune system, improving cognitive function, reducing aging, or improving metabolism, or a combination thereof. The nutritional composition or the nutritional combination may improve, or alleviate a condition or disease, or a plurality of conditions or diseases, including, but not limited to, cardiovascular conditions or diseases, inflammatory conditions or diseases, and metabolic conditions or diseases.

In some embodiments, the nutritional composition alone or nutritional combination improves the immune system or immune function. For non-limiting example, improved immune system or immune function is an enhanced ability to respond to an acute pathogenic stimulus, resulting in an enhanced protective immune response to an exogenous pathogen or to an autologous trigger such as neoplastic cells.

Cardiovascular condition or cardiovascular disease is used herein in the broadest sense includes, without limitation, coronary artery atherosclerosis, coronary microvascular disease, macrovascular disease, stroke, carotid artery disease, peripheral arterial disease, ischemia, coronary artery disease (CAD), coronary heart disease (CHD), conditions associated with CAD and CHI, cerebrovascular disease and conditions associated with cerebrovascular disease, peripheral vascular disease and conditions associated with peripheral vascular disease, aneurysm, vasculitis, venous thrombosis, diabetes mellitus, and metabolic syndrome, chronic kidney disease, remote tissue injury after ischemia and reperfusion, and cardiopulmonary bypass.

Inflammatory condition or inflammatory disease as used herein in the broadest sense includes, without limitation, asthma, chronic obstructive pulmonary disease (COPD), asthma and COPD overlap syndrome (ACOS), atopic dermatitis, nasal polyps, an allergic response, chronic bronchitis, emphysema, chronic rhinosinusitis with or without nasal polyps, inflammatory bowel disease, Crohn's disease, ulcerative colitis, hypersensitivity pneumonitis, multiple sclerosis, arthritis (including osteoarthritis, rheumatoid arthritis, or psoriatic arthritis), allergic rhinitis, fibrosis, eosinophilic esophagitis, vasculitis, urticaria, Churg Strauss syndrome, inflammatory pain and psoriasis.

Metabolic condition or metabolic disease as used herein in the broadest sense includes, without limitation, metabolic syndrome, diabetes type I, diabetes type II, obesity, binge eating, undesired food cravings, food addiction, a desire to reduce food intake or to lose weight or maintain weight loss, desire to maintain healthy weight, desire to maintain normal blood glucose metabolism, anorexia, pre-diabetes, glucose intolerance, gestational diabetes mellitus (GDM), impaired fasting glycemia (IFG), post-prandial hyperglycemia, accelerated gastric emptying, dumping syndrome, delayed gastric emptying, dyslipidemia, post-prandial dyslipidemia, hyperlipidemia, hypertriglyceridemia, post hypertriglyceridemia, insulin resistance, bone loss disorders, osteopenia, osteoporosis, muscle wasting disease, muscle degenerative disorders, polycystic ovary syndrome (PCOS), non-alcoholic fatty liver disease (NAFL), non-alcoholic steatohepatitis (NASH), immune disorders of the gut, (e.g., celiac disease), bowel irregularity, irritable bowel syndrome (IBS), inflammatory bowel disease (IBD), including, e.g., ulcerative colitis, Crohn's disease, short bowel syndrome and peripheral neuropathy, e.g., diabetic neuropathy. In some embodiments, the condition is obesity.

In some embodiments, the nutritional composition alone or nutritional combination, alleviates a condition or disease, or reduces the condition or disease in severity, duration or frequency of occurrence, or alleviates at least one symptom associated with the condition or disease, or reduces in severity, duration, or frequency of occurrence.

The nutritional composition alone or the nutritional combination provides nutrients (e.g. polyphenols, fiber) that may encourage the growth of beneficial microbial species (e.g. Lactobacillus acidophilus) and discourage the growth of less desirable microbial species (e.g. Enterococcus faecalis) in the gut microbiota. Having beneficial microbial species in the gut microbiota provides benefits such as, but not limited to, improving insulin secretion, glucose tolerance and short-chain fatty acid production, while having lower levels of less desirable microbial species helps reduce, for non-limiting example, obesity and inflammation.

### Modes of Delivery, Packaging

The present disclosure relates to nutritional compositions and nutritional combinations that may be modular/component systems of prepared or unprepared food such as, but not limited to, meal replacements, bars, bites, drinks, snacks, meals, desserts, cereals, salad, side dish, sauces, desserts, spreads etc. For example, the nutritional compositions and nutritional combinations can used in prepared or unprepared foods such that consumers can select a specific food or drink item, such as a bottle of juice, bar, a salad, a meal knowing that the nutrients derived from the composition and nutritional combination on the whole will keep them in a safe nutritional or metabolic range. In certain embodiments, such compositions and nutritional combinations are packaged and marked for a particular cohort, such that individuals can conveniently maintain nutritional balance without frequent nutritional counseling.

In other embodiments, the present disclosure relates to a meal replacement or snack comprising the nutritional composition or the nutritional combination. In an aspect, the meal replacement or snack is a nutrition bar or an energy bite.

The nutritional compositions or nutritional combinations according to the present disclosure may also comprise other ingredients that can modify the chemical, physical or processing characteristics of the products or serve as pharmaceutical or additional nutritional components when they are used by certain target populations or cohorts.

Optional ingredients known or otherwise adapted for use in other food products may also be used in the nutritional compositions or nutritional combinations in accordance with the present disclosure, on condition that these optional ingredients are safe and efficient for consumption, administration, and are compatible with the other essential ingredients of the composition.

Non-limiting examples of such optional ingredients comprise buffers, pharmaceutical active agents, additional nutrients, dyes, flavorings, thickeners and stabilizers, etc.

In some embodiments, the nutritional composition or nutritional combination is in powder or liquid form. The nutritional composition or nutritional combination in powder or liquid form may also comprise one or more masking agents to reduce, for example, the bitter tastes in reconstituted powders. Suitable masking agents comprise natural and artificial sweeteners, such as and not limited to, stevia, honey, saccharine, sucralose and aspartame, sources of sodium, such as sodium chloride, and hydrocolloids such as guar gum, xanthan gum, carrageenan, and combinations thereof.

In other embodiments, the nutritional composition comprises a flavorant or flavoring, including and not limited to, chocolate, cocoa, carob powder, cinnamon, mocha or vanilla, or a combination thereof.

In some embodiments, the nutritional composition or nutritional combination does not contain dispersing agents.

In other embodiments, the nutritional composition or nutritional combination does not contain any synthetic color additives.

In other embodiments, the nutritional composition or nutritional combination does not contain one or more of the following sources of empty calories: cellulose gel, maltodextrin, sugar, guar gum, dextrose, erythritol, lactose, sunflower oil, whey, flour, and chemical additives which provide no nutritional benefit such as dicalcium phosphate silicon dioxide, preservatives, and bulking agents.

The nutritional composition or nutritional combination of the present disclosure may be reconstituted with water or another aqueous liquid, including and not limited to, coconut water, dairy milk, nut milk, coffee, fruit or vegetable juice. In other embodiments, the present disclosure relates to the application of these nutritional compositions or nutritional combinations as beverages, for non-limiting example, ready-to-drink beverages, or nutritional liquids, by means of powder mixes to be reconstituted, with water or another aqueous liquid. For non-limiting example, the composition or nutritional combination is reconstituted for dietetic nutrition (sports, slimming), and as ready-to-drink beverages, in which low viscosity of the beverage and an improvement in the solubility of the plant protein are sought. In other embodiments, the ready-to-drink beverage is used for clinical nutrition (oral route or enteral bag) and dietetic nutrition.

The present disclosure also relates to the application of these nutritional compositions or nutritional combinations as dairy or plant-based beverages, in fermented milks of yoghurt type (stirred, Greek or drinking yoghurt) and as dairy or plant-based creams, iced desserts or sorbets.

The present disclosure further relates to the application of these nutritional compositions or nutritional combinations as biscuits, muffins, griddle cakes or nutritional bars (intended for specialized/slimming nutrition or sports nutrition), as protein-enriched breads or gluten-free breads, as high-protein small cereals obtained by extrusion cooking (“crisps”), in which high-protein solutions are more particularly sought without a negative impact on the preparation process or the texture of the preparations or finished products.

The nutritional composition or nutritional combination in powder form is prepared by, for non-limiting example, mixing various powders.

The nutritional composition and booster of the present disclosure may each independently provide an amount of caffeine from natural or synthetic sources. Natural ingredients containing caffeine may be incorporated into the nutritional composition or the booster, such as but not limited to coffee beans and guarana. The nutritional composition or booster of the foregoing may improve energy levels or a person's mood.

### DEFINITIONS

It is to be understood that the terminology used herein is for describing particular embodiments only and is not intended to be limiting. Unless defined otherwise, all technical and scientific terms used herein have the same meaning as commonly understood by one of ordinary skill in the art to which the disclosure pertains.

Although any methods and materials similar or equivalent to those described herein may be used in the practice for testing of the present disclosure, exemplary materials and methods are described herein.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

As used in this specification and the appended claims, the singular forms “a,” “an,” and “the” include plural referents unless the content clearly dictates otherwise. The conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

Unless the context requires otherwise, throughout the specification and claims that follow, the word “comprise” and synonyms and variants thereof such as “have” and “include”, as well as variations thereof, such as “comprises” and “comprising”, are to be construed in an open, inclusive sense, e.g., “including, but not limited to.” The transitional terms “comprising,” “consisting essentially of,” and “consisting of” are intended to connote their generally accepted meanings in the patent vernacular; that is, (i) “comprising,” which is synonymous with “including,” “containing,” or “characterized by,” is inclusive or open-ended and does not exclude additional, unrecited elements or method steps; (ii) “consisting of” excludes any element or step not specified in the claim; and (iii) “consisting essentially of” limits the scope of a claim to the specified materials or steps “and those that do not materially affect the basic and novel characteristic(s)” of the claimed invention and disclosure. Embodiments described in terms of the phrase “comprising” (or its equivalents) also provide as embodiments those independently described in terms of “consisting of” and “consisting essentially of.”

“About” means within an acceptable error range for the particular value as determined by one of ordinary skill in the art, which will depend in part on how the value is measured or determined, i.e., the limitations of the measurement system. Unless explicitly stated otherwise within the disclosure, claims, result or embodiment, “about” means within one standard deviation per the practice in the art, or can mean a range of ±20%, ±10%, ±5%, ±4, ±3, ±2 or ±1% of a given value. It is to be understood that the term “about” can precede any particular value specified herein, except for particular values used in the Examples.

All percents are intended to be weight percent (on a dry weight basis) unless otherwise specified. The present disclosure is not to be limited in scope by the specific embodiments described herein. Indeed, other various embodiments of and modifications to the present disclosure, in addition to those described herein, will be apparent to those of ordinary skill in the art from the foregoing description and accompanying drawings. Thus, such other embodiments and modifications are intended to fall within the scope of the present disclosure. Further, although the present disclosure has been described herein in the context of a particular implementation in a particular environment for a particular purpose, those of ordinary skill in the art will recognize that its usefulness is not limited thereto and that the present disclosure may be beneficially implemented in any number of environments for any number of purposes. Accordingly, the claims set forth below should be construed in view of the full breadth and spirit of the present disclosure as described herein.

### EXEMPLIFICATION

Elevated cholesterol and triglycerides and reduced peripheral vascular health contribute to increased risk for cardiovascular (CVD) and metabolic disease and elevated oxidative stress and/or inflammation contribute.

Purpose: This study tested the hypothesis that daily consumption of a beverage high in antioxidants, polyphenols, and anti-inflammatory properties would improve blood lipid profile and well-validated indices of peripheral vascular function.

METHODS: Six individuals (4 males; age: 42±14 yr.; BMI 32±7 kg/m²) participated in this preliminary dataset. Measures were assessed before (pre) and 2 & 6-weeks following (post) consumption of the GOODE HEALTH® Superfood Moroccan Chocolate Blend (Goode Health LLC, Stamford, CT) and the following boosters: 1) metabolism booster containing berberine, ginger, and cinnamon, 2) immune support booster containing bilberry, green tea extract, isoquercetin, echinacea, vitamin C, and zinc, 3) vascular booster containing black garlic, bergamot, and flax seeds, and 4) recovery booster containing turmeric with MERIVA™ bioavailable coating and isoquercetin, in which ˜28 g of the blend powder was added to 8 oz water twice daily. This beverage is high in plant-based proteins, prebiotic fiber, antioxidant polyphenols, and anti-inflammatory properties. Blood was collected via venipuncture and was analyzed for basal lipid profile. Peripheral macro and microvascular function/health were assessed as vasodilation in the brachial artery (flow mediated vasodilation; % FMD) and reactive hyperemia (peak mean forearm blood velocity (FBVmean)) in the forearm vasculature respectively following 5-min of forearm ischemia.

RESULTS: Lipid profile was improved following 2-weeks of GOODE HEALTH® beverage consumption (Total-C, Pre: 182±30 vs. Post: 155±30 mg/dl, P=0.10; VLDL, Pre: 14±5 vs. Post: 12±4 mg/dl, P=0.08; LDL Pre: 121±24 vs. Post: 98±25 mg/dl, P=0.13; TG Pre: 77±30 vs. Post: 58±26 mg/dl, P=0.01). % FMD tended to be augmented at 2-weeks (Pre: 4.2±2.2 vs. Post: 5.8±1.6%, P=0.08) and 6-weeks (Post: 7.2±0.4%, P=0.06 compared to Pre) of beverage consumption. FBVmean was or tended to be augmented following beverage consumption (2-weeks Pre: 70±17 vs. Post: 78±20 cm·s⁻¹, P=0.04; 6-weeks Post: 89±22 cm·s⁻¹, P=0.06 compared to Pre).

CONCLUSION: Preliminary data indicate that the nutritional composition results in a positive effect on blood lipid markers in subjects with respect to a control group. Cholesterol measures also displayed significant improvement, with total cholesterol decreasing by 14.5%, LDL cholesterol reducing by 18.7%, and Triglycerides dropping by 25.4%. A crucial marker of immune health & inflammation, C-reactive Protein, decreased by about 6% after two weeks of GOODE HEALTH® beverage consumption. These preliminary data indicate a positive effect of GOODE HEALTH® Superfood Moroccan Chocolate Blend on indices of lipid profile and vascular function/health.

1. A high-density nutritional composition providing a serving size of from about 25 g to about 60 g in dry weight, comprising:
nutrients comprising protein, polyphenol, fiber, vitamins, and minerals, wherein:
the protein is present in an amount of from about 5 g to about 35 g;
the polyphenol is from at least one source to collectively provide a level of polyphenols of from about 50 mg to about 200 mg;
the fiber is present in an amount of from about 2.5 g to about 24 g;
the vitamins and minerals are present in an amount of from about 0 g to about 6 g; and
the nutrients do not contribute to empty calorie content of the formulation, and filler comprising carbohydrate and fat, wherein:
the carbohydrate is present in an amount of from about 1 g to about 15 g;
the fat is present in an amount of from about 0 g to about 6 g,
wherein the high-density nutrient composition has a ratio of the nutrients to the filler that exceeds about 1.5:1 on a dry weight basis.

2. A high-density nutritional composition, comprising:
nutrients comprising protein, polyphenol, fiber, vitamins, and minerals; wherein:
the protein is present in an amount of from about 20 wt. % to about 60 wt. %;
the polyphenol is from at least one source to collectively provide a level of polyphenols of from about 0.1 wt. % to about 0.7 wt. %;
the fiber is present in an amount of from about 10 wt. % to about 40 wt. %;
the vitamins and minerals are present in an amount of from about 0 wt. % to about 10 wt. %;
the nutrients do not contribute to empty calorie content of the formulation, and filler comprising carbohydrate and fat, wherein:
the carbohydrate is present in an amount of from about 3 wt. % to about 25 wt. %;
the fat is present in an amount of from about 0 wt. % to about 10 wt. %,
wherein the high-density nutrient composition has a ratio of the nutrients to the filler that exceeds about 1.5:1 on a dry weight basis.

3. The composition of claim 2, wherein the level of polyphenols is from about 0 wt. % to about 1 wt. %.

4. The composition of claim 1, wherein the composition provides calories in an amount of from about 2.5 calories per gram of composition to about 3.5 calories per gram of composition.

5. The composition of claim 1, wherein the composition provides less than about 0.5 grams of sugar.

6. The composition of claim 1, wherein the composition provides less than about 0.5 grams of saturated fat or less than about 0.5 grams of trans fat.

7. The composition of claim 1, wherein the composition further comprises vitamin B9, zinc, vitamin A, magnesium, vitamin C, vitamin E, potassium, vitamin D, iron, or calcium, or a combination thereof.

8. The composition of claim 1, wherein the polyphenols comprise flavonoids or phenolic acids, or a combination thereof.

9. The composition of claim 1, wherein the sources of plant polyphenols comprise bilberry, green tea, isoquercetin, echinacea, carob, or monk fruit, or a combination thereof.

10. The composition of claim 1, further comprising a flavorant, wherein the flavorant is a vanilla or chocolate flavorant.

11. The composition of claim 1, wherein the carbohydrate is one or more of starch, sugar, or gum, or a combination thereof.

12. A nutritional combination, comprising the composition of claim 1 and an antioxidant booster.

13. The nutritional combination of claim 12, wherein the booster comprises bilberry, green tea extract, isoquercetin, echinacea, vitamin C, and zinc.

14. The nutritional combination of claim 12, wherein the booster comprises berberine, ginger, and cinnamon.

15. The nutritional combination of claim 12, wherein the booster comprises black garlic, bergamot, and flax seeds.

16. The nutritional combination of claim 12, wherein the booster comprises turmeric and isoquercetin.

17. The composition of claim 2, wherein the composition is in a powder or liquid form, and optionally comprises an antioxidant booster.

18. The composition of claim 17, wherein the composition is reconstituted with water or another aqueous liquid.

19. A meal replacement or snack comprising the composition of claim 1 and an optional antioxidant booster.

20. The meal replacement or snack of claim 19, wherein the meal replacement or snack is a nutrition bar or an energy bite.

21. A beverage comprising the composition of claim 1 and an optional antioxidant booster.

22. The beverage of claim 21, wherein the beverage is a ready-to-drink beverage.

23. A method of improving vascular health, reducing inflammation, enhancing immune system, or improving metabolism, or a combination thereof, comprising administering the composition according to claim 1 and an optional antioxidant booster, to a subject in need thereof.

24. The method of claim 23, wherein improving vascular health comprises improving one or more of blood circulation, blood vessel flexibility, and blood pressure.

25. A method of improving cognitive function or reducing aging, comprising administering the composition according to claim 1 and an optional antioxidant booster, to a subject in need thereof.
